(19) United States
(12) Patent Application Publication
(10) Pub. No.: US 2014/0377389 A1
(43) Pub. Date: Dec. 25, 2014

(54) POMEGRANATE EXTRACT FOR USE IN TREATING RHINITIS AND SINUSITIS

(71) Applicant: GANIR (1992) LTD., Mobile Post Sde Gat (IL)

(72) Inventors: Eli Hugo Budman, Mobile Post Hof Carmel (IL); Arie Yaari, Nahariya (IL); Itzhak Braverman, Nesher (IL)

(73) Assignee: GANIR (1992) LTD., Mobile Post Side Gat (IL)

(21) Appl. No.: 14/382,867

(22) PCT Filed: Mar. 6, 2013
(86) PCT No.: PCT/IL2013/050199

§ 371 Date: Sep. 4, 2014

Related U.S. Application Data
(60) Provisional application No. 61/607,645, filed on Mar. 7, 2012.

(51) Int. Cl. A61K 36/185; A61K 45/06; A61K 9/00

(52) U.S. Cl. CPC A61K 36/185; A61K 9/0043; A61K 45/06; A61K 2236/19
USPC 424/769

(57) ABSTRACT

This invention provides a composition comprising a pomegranate extract, formulated as nasal drops or nasal spray, for use in treating the symptoms of rhinitis or sinusitis.

### FIELD OF THE INVENTION

The present invention relates to a composition for treating sinusitis or rhinitis. The composition comprises a pomegranate extract, and optionally other agents, and may be formulated as nose drops or as nose spray.

### BACKGROUND OF THE INVENTION

Rhinitis and sinusitis have complex etiology, and more mechanisms contribute to their symptoms. Typical symptoms of acute rhinitis, physically unpleasant and socially annoying, include nasal discharge and obstruction, and they often result from common cold and other viral infections, but they may also be caused by a variety of bacteria, like streptococci, pneumococci, or staphylococci. Some types of rhinitis lead to severe changes in the mucous membrane and prolonged suffering, marked by periods of remission and exacerbation. Chronic rhinitis may be an accompanying symptom of grave systemic diseases. Causative therapy like systemic antibiotics, or symptomatic treatment including vitamins or topical vasoconstrictors are used, but their efficiency may be limited. Some types of rhinitis have no known cause, like atrophic rhinitis (The Merck Manual, 17th Ed., p. 685, 1999). Rhinitis may be aggravated by polyp formation, usually treated by corticosteroids, sometimes it is operated. Non-allergic rhinitis may be triggered by smells, fumes, smoke, dusts, and temperature changes. Allergic rhinitis, whether caused by clearly defined allergens like pollens or not, is treated by topical use of antihistamines or decongestants, and sometimes by glucocorticoids. Sinusitis, inflammation of the paranasal sinuses, may have similar causes as rhinitis, but the symptoms include a strenuous headache or toothache, rather than nasal discharge. Usual treatments again include antibiotics and topical vasoconstritors, and possibly an operation. Aggressive microbially caused sinusitis may be even fatal in compromised patients, like diabetic or immunodeficient persons [The Merck Manual, 17th Ed., p. 688, 1999].

Newer classifications of sinusitis refer to it as rhinosinusitis, taking into account the thought that inflammation of the sinuses cannot occur without some inflammation of the nose as well (rhinitis). Rhinitis and sinusitis are among the most common medical conditions in Western societies: an estimated 10% to 25% of the population have allergic rhinitis, with 30 to 60 million persons being affected annually in the United States, while sinusitis affects 31 million patients annually in the United States; both rhinitis and sinusitis can significantly decrease quality of life, aggravate comorbid conditions, and require significant direct medical expenditures [Dykewicz M. S. and Hamilos D. L.: Rhinitis and sinusitis. J. Allergy Clin. Immunol. 125 (2 Suppl 2), 2010 Feb, S103-15].

Both rhinitis and sinusitis, although usually comprising inflammatory features, often lack a clear cause, and often are treated by the above traditional means trying to affect the symptoms, but the attempts are often unsuccessful, and the available medicaments have undesired side effects. It is therefore an object of this invention to provide a new means for alleviating irksome symptoms of rhinitis and sinusitis.

It is another object of this invention to provide a composition for topical use for mitigating the symptoms of rhinitis and sinusitis.

Other objects and advantages of present invention will appear as description proceeds.

### SUMMARY OF THE INVENTION

The invention provides a composition for treating the symptoms of rhinitis or sinusitis comprising a pomegranate extract. In one embodiment, said symptoms may be associated with chronic rhinitis, acute rhinitis, virus associated rhinitis, bacteria associated rhinitis, vasomotor rhinitis, rhinitis with polyps, atrophic rhinitis, allergic rhinitis, hay fever, or perennial rhinitis. In other embodiment, said symptoms may be associated with chronic sinusitis, acute sinusitis, viral sinusitis, bacterial sinusitis, fungal sinusitis, and allergic sinusitis.

The invention provides a method of treating rhinitis or sinusitis, comprising administering a composition comprising a pomegranate extract to a patient in need of such treatment. The invention further provides a method of alleviating symptoms of rhinitis or sinusitis, comprising administering a composition comprising a pomegranate extract to a patient suffering from the said symptoms. In one embodiment, the method according to the invention comprises a patient who is indicated for a polyp removal or for sinusitis operation.

The invention provides a composition comprising a pomegranate extract, formulated as nasal drops or nasal spray. The composition may further Comprise, beside the pomegranate extract, an additive selected from the group consisting of diluent, stabilizer, and at least one additional agent selected from analgesic, antiviral, antibacterial, antifungal, vasoconstrictor, antihistamine, decongestant, anti-inflammatory, and corticosteroid. The composition according to the invention comprises polyphenols, preferably in an amount of from 100 ppm to 40,000 ppm (according to Folin Ciocalteu method). The composition according to the invention comprises punicalagins, preferably in an amount of from 100 ppm to 20,000 ppm (by HPLC).

The invention relates to a method of producing the composition comprising a pomegranate extract for treating the symptoms of rhinitis or sinusitis, comprising extracting pomegranate fruit or a pomegranate component, thereby obtaining a pharmaceutically active extract which is diluted or otherwise formulated to attain the level of polyphenols (according to Folin Ciocalteu method) of from 100 ppm to 40,000 ppm, and/or to attain the level of punicalagins (by HPLC) of from 100 ppm to 20,000 ppm. In one embodiment, said method further comprises lowering the amount of available sugars, possibly by fermenting said extract or by nanofiltration and/or using other membrane technologies, thereby obtaining an active extract with lowered amount of sugars. The method according to the invention may comprise extracting pomegranate fruit or a part of pomegranate tree, such as leaves, peel, flowers, husks, membranes, pulp, seeds or seeds fractions, and mixtures thereof. The method may comprise extracting pomegranate fruit with hot water, cold water, or organic solvents such as ethanol or essential oils.

The invention provides a pomegranate extract for topical use in mitigating the symptoms of rhinitis and sinusitis, comprising polyphenols (according to Folin Ciocalteu method) in an amount of from 100 ppm to 40,000 ppm, and/or punicalagins (by HPLC) in an amount of from 100 ppm to 20,000 ppm.

The invention provides a composition comprising a pomegranate extract for treating the symptoms of rhinitis and sinusitis, both chronic or acute, including, among others, virus associated rhinitis, bacteria associated rhinitis, vasomotor rhinitis, rhinitis with polyps, atrophic rhinitis, allergic rhinitis, hay fever, perennial rhinitis, viral or microbial sinusitis, and allergic sinusitis.

### DETAILED DESCRIPTION OF THE INVENTION

It has now been found that a processed and diluted extract from pomegranate fruits alleviates symptoms of rhinitis as well as symptoms of sinusitis. The extract may be applied in a composition comprising other components.

The composition according to the invention is preferably prepared by extracting pomegranate fruit or a pomegranate component, thereby obtaining a pharmaceutically active extract which may be diluted or otherwise formulated. The process of preparing the composition further preferably comprises lowering the amount of available sugars, possibly by fermenting said extract or by nanofiltration and/or using other membrane technologies, thereby obtaining an active extract with lowered amount of sugars.

The pharmaceutical composition according to the invention may comprise a pomegranate extract and an additive selected from the group consisting of diluent, stabilizer, and at least one additional agent selected from analgesic, antiviral, antibacterial, antifungal, vasoconstrictor, antihistamine, decongestant, anti-inflammatory, and corticosteroid.

The extract for use according to the invention may comprise pomegranate fruit or a part of pomegranate tree, such as leaves, peel, flowers, husks, membranes, pulp, seeds or seeds fractions, and mixtures thereof. The extract can be applied alone or combined with other agents, either natural extracts or medicaments.

The pomegranate fruit may be extracted by aqueous extraction, with hot or cold water, or in another embodiment by the extraction with solvents, such as ethanol or essential oils. The extraction may be performed under higher than normal pressure. The extraction methods may include decanting, centrifuging, filtering through membranes, adsorbing, absorbing (using, for example, diatomaceous earth or carbon), chromatographing, evaporating, and other separation and purification techniques. The employed physical methods may utilize decanters, finishers, centrifuges, filters, sorption resins, ion exchange resins, evaporators, and other devices.

In a preferred embodiment, the process for the preparation of the pomegranate-based pharmaceutical means includes removing sugars from the pomegranate fractions or extract before the medical use. Said removing sugars may employ physical, chemical or biological methods like nanofiltration and fermentation.

The extract can be modified by techniques used in preparative biochemistry, including enzyme utilization, in order to enrich a specific fraction or to obtain a desired ratio of different fractions. The extract or fractions for the use in a method of the invention can be obtained, for example, by aqueous extraction, by water under pressure, by solvents, ethanol, essential oils, and/or by physical methods like filtration through membranes, adsorption by resins, or ion exchange resins, or by combinations of them. The extraction process may include removal of sugars by physical, chemical or biological methods like nanofiltration and fermentation.

Undesired pomegranate components may be removed during the preparation of an active extract, other components may be concentrated. Various pomegranate pytochemicals may be concentrated by separation and recomposition, including polyphenols. In one aspect of the invention, a pomegranate extraction process comprises a treatment by a sorption resin which absorbs active components, which can be later released and utilized in the final composition. For example, polyphenols or other components are absorbed in resins and then recovered into water. Compositions may be sterilized by irradiating by gamma or autoclaved. The active components or fractions may be formulated, for example, as nasal spray or nasal drops.

Without wishing to be limited by any particular theory, the inventors believe that the efficacy of the composition of the invention may be related to the fact that the pomegranate extract is a complex mixture of many natural components, of which some were showed to have various biologically relevant activities, such as antimicrobial, antioxidative, etc. As both rhinitis and sinusitis are of complex and often unclear etiology, a combination of natural components present in the extract or diluted extract according to the invention may interfere with the cascade of events associated with the formation of said symptoms, resulting in mitigating said symptoms, and possibly in assisting the body in full recuperation.

In one aspect of the invention, either rhinitis or sinusitis is treated by applying nose drops comprising a pomegranate extract. In a preferred embodiment of the method according to this invention, sinusitis is treated by spraying an aerosol comprising pomegranate extract into the nose. In other preferred aspect of the invention, rhinitis is treated by spraying an aerosol comprising pomegranate extract into the nose.

The extract is mixed with pharmaceutically acceptable components and formulated as nose drops or as a nose spray. The extract, in a preferred embodiment of the invention, is diluted before the use with sterile water. The final composition contains only pharmaceutically acceptable solvents and components.

The invention will be further described and illustrated by the following examples.

### EXAMPLES

### Example 1

The whole fruit was homogenized, the amount of sucrose was reduced by fermentation, the mixture was centrifuged and, after resin treatment, sterilized at 120° C. for 20 minutes. The clear supernatant was used as nose drops. One adult person suffering from acute rhinitis and one adult person suffering from acute sinusitis were provided the drops, in a dripping bottle making drops of about 0.15 ml. The persons dripped one drop to each nostril every morning and every evening. The person with rhinitis reported improvement of the symptoms after two days; the person with sinusitis reported alleviation after four days.

### Example 2

A controlled clinical trial will now be performed, including four groups of adult people, 20 people in each group. Groups A and B will comprise patients suffering from chronic rhinitis, groups C and D from chronic sinusitis; groups A and C will be treated with the composition according to the invention, groups B and D with placebo. The composition according to the invention will comprise an pomegranate extract, treated with a sorbent resin, with lowered sugars content and increased polyphenol content. The y-irradiated composition, stored at 4° C. will be used as nose drops, one drop into each nostril twice a day. A sterile aromatized physiological solution will serve as a placebo. The effects will be evaluated after 45 days, taking special attention to distinguishing patients with and without polyps, and patients indicated for the sinusitis operation. Before and after the treatment, among others, the following examination will be performed: throat, nose, endoscopic examination, air flow, and eosinophils level. The patients indicated for the operation will be CT examined.

### Example 3

Compositions comprising pomegranate fractions, salt (sodium chloride), preservatives and other excipients were prepared.

In one case, a pomegranate-derived powder containing 30% punicalagins (determined by HPLC) and 60% total polyphenols (according to Folin Ciocalteu method) was used. In order to obtain a long shelf life at room temperature, the composition was formulated as a kit comprising two separate components: (a) a solid component essentially consisting of a pomegranate-derived powder, rich in punicalagins and polyphenols, and containing the salt and other excipients; and (b) a liquid fraction essentially consisting of sterile water. Of course, the salts and other excipients may be combined with water and sterilized, thereby providing component (b), while said component (a) may comprise less salts and excipients or none. The dry fraction contained 300 mg of pomegranate-derived powder, 275 mg of sodium chloride and buffer salts providing, after dissolution in 15 ml sterile water, final nasal spray or nasal drops containing about 0.6 wt % of punicalagins, about 1.2 wt % total polyphenols, and a final pH of about 5.3. The final solution was isotonic with body fluids. The final solution for nasal drops or nasal spray was stored at 4° C. The level of ellagic acid, punicalagins, and total polyphenols was checked during 8 weeks, as shown in the table:

| Ellagic acid | | Total polyphenols | | Punicalagins | | Number of |
|---|---|---|---|---|---|---|
| % | PPM | % | PPM | % | PPM | weeks |
| 100 | 287 | 100 | 10643 | 100 | 6083 | 0 |
| 100 | 287 | 96 | 10171 | 104 | 6318 | 2 |
| 96 | 274 | 101 | 10795 | 108 | 6581 | 4 |
| 92 | 264 | 106 | 11262 | 102 | 6184 | 8 |

The levels of punicalagins and polyphenols in the final nasal drops or nasal spray remained stable during 8 weeks of storage at 4° C.

It was found that the levels of punicalagins and polyphenols remained stable even during 1 year of storage at room temperature, when the two components of the composition were stored separately.

While the invention has been described using some specific examples, many modifications and variations are possible. It is therefore understood that the invention is not intended to be limited in any way, other than by the scope of the appended claims.

1. A composition for treating the symptoms of rhinitis or sinusitis comprising a pomegranate extract.

2. A composition according to claim 1, wherein said symptoms are associated with chronic rhinitis, acute rhinitis, virus associated rhinitis, bacteria associated rhinitis, vasomotor rhinitis, rhinitis with polyps, atrophic rhinitis, allergic rhinitis, hay fever, or perennial rhinitis

3. A composition according to claim 1, wherein said symptoms are associated with chronic sinusitis, acute sinusitis, viral sinusitis, bacterial sinusitis, fungal sinusitis, and allergic sinusitis.

4. A method of treating rhinitis or sinusitis, comprising administering a composition according to claim 1 to a patient in need of such treatment.

5. A method of alleviating symptoms of rhinitis or sinusitis, comprising administering a composition according to claim 1 to a patient suffering from said symptoms.

6. A method according to claim 4, wherein said patient is indicated for a polyp removal or for sinusitis operation.

7. A composition according to claim 1, formulated as nasal drops or nasal spray.

8. A composition according to claim 1, comprising a pomegranate extract and an additive selected from the group consisting of diluent, stabilizer, and at least one additional agent selected from analgesic, antiviral, antibacterial, antifungal, vasoconstrictor, antihistamine, decongestant, anti-inflammatory, and corticosteroid.

9. A composition according to claim 1, comprising polyphenols (according to Folin Ciocalteu method) in an amount of from 100 ppm to 40,000 ppm.

10. A composition according to claim 1, comprising punicalagins (by HPLC) in an amount of from 100 ppm to 20,000 ppm.

11. A method of producing the composition of claim 1, comprising extracting pomegranate fruit or a pomegranate component, thereby obtaining a pharmaceutically active extract which is diluted or otherwise formulated to attain the level of polyphenols (according to Folin Ciocalteu method) of from 100 ppm to 40,000 ppm, and/or to attain the level of punicalagins (by HPLC) of from 100 ppm to 20,000 ppm.

12. A method according to claim 11, further comprising lowering the amount of available sugars, possibly by fermenting said extract or by nanofiltration and/or using other membrane technologies, thereby obtaining an active extract with lowered amount of sugars.

13. A method according to claim 11, comprising extracting pomegranate fruit or a part of pomegranate tree, such as leaves, peel, flowers, husks, membranes, pulp, seeds or seeds fractions, and mixtures thereof.

14. A method according to claim 11, comprising extracting pomegranate fruit with hot water, cold water, or organic solvents such as ethanol or essential oils.

15. A pomegranate extract for topical use in mitigating the symptoms of rhinitis and sinusitis, comprising polyphenols (according to Folin Ciocalteu method) in an amount of from 100 ppm to 40,000 ppm, and/or punicalagins (by HPLC) in an amount of from 100 ppm to 20,000 ppm.
